(19) United States
(12) Patent Application Publication
(10) Pub. No.: US 2010/0266560 A1
(43) Pub. Date: Oct. 21, 2010

(54) AGENT STABILISATION PROCESS AND PRODUCT

(75) Inventors: Jayanthi Swaminathan, Christchurch (NZ); Trevor Anthony Jackson, Christchurch (NZ)

Correspondence Address: SUGHRUE MION, PLLC, 2100 PENNSYLVANIA AVENUE, N.W., SUITE 800, WASHINGTON, DC, 20037, US

(73) Assignee: ENCOATE HOLDINGS LIMITED, Hamilton (NZ)

(21) Appl. No.: 12/741,984

(22) PCT Filed: Nov. 6, 2008
(86) PCT No.: PCT/NZ08/00299

§ 371 Date: May 7, 2010

(30) Foreign Application Priority Data
Nov. 7, 2007 (NZ) 560574

(51) Int. Cl. A61K 35/74; C12N 1/04; A23L 1/30; B05D 1/36

(52) U.S. Cl. 424/93.44; 435/260; 424/93.4; 424/93.45; 424/93.47; 426/61; 427/402

(57) ABSTRACT

The invention relates to a composition and method of manufacture including a substrate coated with a biopolymer and aqueous biological gel and subsequently coated with at least one desiccation agent. The resulting composition is dry to touch, has a low water activity and stabilises the biological material for storage over at least one month at ambient temperatures.

### STATEMENT OF CORRESPONDING APPLICATIONS

This application is based on the Provisional specification filed in relation to New Zealand Patent Application Number 560574, the entire contents of which are incorporated herein by reference.

### TECHNICAL FIELD

The invention relates to an agent stabilisation process and product. More specifically, the invention relates to an alternative method to stabilise biological materials as well as to produce a product ready for delivery.

### BACKGROUND ART

A known problem associated with the industrial or agricultural application of biological materials is the maintenance of the materials in a viable state or a stable state until they are used, or during the period of time required to stabilise the material such as before drying. Many biological materials cannot be maintained in a viable condition during storage, particularly where they are not kept or cannot be kept under refrigeration.

For the purpose of this specification the term ‘biological material’ is used to encompass, but is not limited to, any or all of the following: a micro-organism, biological cells, a part or parts of biological cells, attenuated micro-organisms, spores, mycelia, including hypha, pharmaceutical compounds unstable at room temperature, enzymes, hormones, proteins, and combinations thereof. For the purpose of the discussion following, specific mention is made towards bacteria but as noted above, should not be seen as limiting.

At present, use of bacterial products as the biological material requires production of high concentrations of bacteria to ensure survival of commercially useful numbers by the time the product is used. The term ‘shelf life’ refers to the storage time period post processing, but it should be appreciated that the need to ensure survival of the bacteria starts with the raw material and is maintained throughout the processing stages. This has been achieved to a limited degree using chilling during before, during and after processing ('cold chain') and/or freeze drying to preserve viability. In additional, while some microbial products require only the delivery of an inoculative dose, for others (such as bio-pesticides), delivery of a higher minimum dosage concentration is essential to delivery of an efficacious dose.

A number of different formulations and media have been proposed, used and disclosed in order to overcome this shelf-life problem. Some formulations emphasise the selection of the basic active ingredient for the storage matrix ‘the bio-polymer’, whilst others disclose methods for preparation of this matrix, or the method of introduction of the biological agent into the matrix and the conditions under which any of these steps occur.

One method used to stabilise agents is to mix the agent or agents with a polysaccharide carrier such as a wax, starch or gum. Whilst this method may address the stability of the agent or agents, the inventors have found that it may not always address dispersion issues and form homogenous results.

The applicant's previous patent application published as WO 02/15702, incorporated herein by reference, describes a method of producing a stable bio-matrix gel. Whilst this is useful in providing a stabilised agent, a gel is not always the preferred delivery mechanism. The application also describes spreading the gel into a 5 mm thick layer and then drying. The inventor's experience is that this thickness can be slow to dry and is mainly appropriate for delivery where the dried gel is re-hydrated and thoroughly agitated before use. Milder forms of mixing may be insufficient to fully re-hydrate and homogenise the agent into solution, particularly when dissolution needs to occur relatively quickly.

A further patent application by the applicant published as WO 02/15703 describes an extension to the WO02/15702 method whereby the bio-matrix gel is further mixed with powdered inert clay to form a dough. The dough is described as being formed into granules or pellets which may then be dried. Similar drying issues may occur in this case where thicker granules and/or pellets are slower to dry than a thin film and are mainly appropriate for delivery where the dried dough is re-hydrated and thoroughly agitated. Milder forms of mixing may be insufficient to fully re-hydrate and homogenise the agent into solution, particularly when dissolution needs to occur relatively quickly. One problem partially addressed in this application is delivery of the agent directly with a vehicle such as a seed. An example is provided where the dried dough is re-hydrated in water and then seeds are dipped into the solution and drilled into the ground. Disadvantages of this method though include the need to perform a re-hydration step before drilling as this introduces a further labour requirement as well as an opportunity for the biological material to degrade once hydrated. Ideally it would be useful to have the seed or other vehicle ready for use without needing this hydration and immersion step.

Methods disclosed in WO 02/15703 also include drying which increases the labour required (and processing costs) and which is consequently undesirable.

It should be appreciated by those skilled in the art that storage stability is important. Also of importance is the need to provide the stabilised biological material in a form ready and easy for subsequent use. It is preferable that the agent not only be stabilised, but also be prepared in a form ready for use in desired applications with minimum preparation and processing expense.

It is an object of the present invention to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### DISCLOSURE OF THE INVENTION

According to one aspect of the present invention there is provided a composition including:
(a) a substrate;
(b) a first coating that at least partially coats the surface of the substrate including: at least one gum based biopolymer; and an aqueous concentrate of biological material; and,
(c) a second coating that at least partially coats the surface of the first coating including at least one desiccation agent.

According to a further aspect of the present invention, there is provided a method of producing a composition including stabilised biological material and a substrate, by the steps of:
(a) mixing at least one substantially dry and powdered biopolymer with an aqueous concentrate of biological material to form a gel;
(b) coating the gel formed in step (a) as a first coating onto at least part of the surface of the substrate material to form a gel coated substrate ('first coating'); and,
(c) at least partially coating the first coating with at least one desiccation agent (‘second coating’).

According to a further aspect of the present invention there is provided a food including a composition substantially as described above.

Preferably, the food may be substantially dry and stored at ambient temperature.

According to a further aspect of the present invention there is provided a nutraceutical product including a composition substantially as described above.

According to a further aspect of the present invention there is provided a food ingredient including a composition substantially as described above.

The invention broadly relates to a double coated substrate which is ready for use in that the substrate and biological material are in one composition. The initial biological material is fresh and in an aqueous state and the process provides a method of reducing the water activity of the environment around the biological material and thereby providing the desired degree of stability/viability when stored over time. In the invention this is achieved using desiccation agents rather than prior art drying methods. In addition to the composition having superior stability over prior art methods, the invention is also easy to process being simple and requiring minimal processing steps and equipment.

In selected embodiments the composition and method may include addition of at least one further layer on earlier layers wherein the further layer includes at least one desiccant.

For the purposes of this specification, the term ‘stable’ or grammatical variations thereof refers to a biological viability of less than 2 log loss in viability when the composition is stored for at least 1 month at 20° C. Preferably, this stability measure relates to the composition when stored in a sealed environment although oxygen may be present in the environment. In further embodiments the loss in viability is no more than 1 log loss.

More preferably, the stability observed may be for time periods in excess of 3 months. In one embodiment, the biological material is stable for over 7 months when stored at 20° C.

Preferably, the second coating may act to reduce the water activity of the first coating.

Preferably, the composition after step (c) may have a water activity of less than 0.7. More preferably, the water activity is less than 0.5. In further embodiments, the water activity may be less than 0.4.

Preferably, the composition after step (c) may be dry to touch.

Preferably, the gel used to form the first coating in step (b) may be a non-Newtonian pseudoplastic fluid. More preferably, the gel may also have thixotropic properties.

Preferably, the biopolymer gum used in step (a) may be characterised by having a molecular weight of between 5000 and 50 million. The biopolymer gum may also be characterised by being resistant to enzymatic degradation as well as being resistant to shear, heat, and UV degradation. In preferred embodiments, the gum when mixed in the composition confers pseudoplastic properties to gels produced.

More specifically, the biopolymer gum may be selected from: agar, alginate, cassia, dammar, pectin, beta-glucan, glucomannan, mastic, chicle, psyllium, spruce gum, xanthan gum, gellan gum, acacia gum, guar gum, locust bean gum, carrageenans, gum arabic, karaya gum, ghatti gum, tragacanth gum, konjac gum, tara gum, and combinations thereof.

In a preferred embodiment the gum may be xanthan gum, gellan gum, locust bean gum, guar gum, and combinations thereof.

In preferred embodiments, the concentration of biopolymer or biopolymers in the composition after step (c) may be approximately 1% to 10% by weight of biopolymer gum. In a more preferred embodiment, the range may be 2% to 6%. In a still more preferred embodiment, the range may be 3% to 5%.

Preferably, the biopolymer gum may have a particle size less than approximately 2 mm in diameter at step (a) before mixing. In one embodiment, the particle size may be approximately 20 mesh or less than 850 μm, although this should not be seen as limiting.

Preferably, the biological material may be bioactive such that it may have an interaction with cell tissue.

Preferably, the biological material used in step (a) may be: a micro-organism, biological cells, a part or parts of biological cells, attenuated micro-organisms, spores, mycelia including hypha, enzymes, hormones, proteins, and combinations thereof.

In a further embodiment, the biological material may be one or more pharmaceutical compounds such as hormones unstable at ambient temperatures.

Preferably, the biological material provided initially may be an aqueous concentrate. In one embodiment, the biological material may be fresh being a culture or concentrate produced within 24 hours of commencing the method of the present invention. The concentrate has not been pre-dried or otherwise processed before stabilising commences in the invention method.

Preferably, the aqueous concentrate may include biological material ranging in concentration from approximately 5% to 99.9% by weight with the remaining content being water or other liquids.

In one embodiment the biological material may be gram negative bacteria.

In an alternative embodiment, the biological material may be gram positive bacteria.

In an alternative embodiment, the biological material may be obligate anaerobe bacteria.

In selected embodiments, the biological materials may be selected from the genus: Serratia, Xanthamonas, Pseudomonas, Rhizobium, Beauveria, Metarhizium, Yersinia, Trichoderma, and combinations thereof.

In alternative embodiments, the biological material may be probiotic bacteria or fungi. For the purposes of this specification, the term ‘probiotic’ refers to viable bacteria and fungi such as yeasts that beneficially influence the health of the host.

Probiotic bacteria include those belonging to the genera Lactococcus, Streptococcus, Pediococcus, Enterococcus, Leuconostoc, Carnobacterium, Propionibacterium, Lactobacillus or Bifidobacterium.

Bifidobacteria used as probiotics include Bifidobacterium adolescentis, Bifidobacterium bifidum, Bifidobacterium animalis, Bifidobacterium thermophilum, Bifidobacterium breve, Bifidobacterium longum, Bifidobacterium infantis and Bifidobacterium lactis. Specific strains of Bifidobacteria used as probiotics include Bifidobacterium breve strain Yakult, Bifidobacterium breve R070, Bifidobacterium lactis Bb12, Bifidobacterium longum R023, Bifidobacterium bifidum R071, Bifidobacterium infantis R033, Bifidobacterium longum BB536 and Bifidobacterium longum SBT-2928.

Lactobacilli used as probiotics include Lactobacillus acidophilus, Lactobacillus brevis, Lactobacillus bulgaricus, Lactobacillus casei, Lactobacillus cellobiosus, Lactobacillus crispatus, Lactobacillus curvatus, Lactobacillus fermentum, Lactobacillus GG (Lactobacillus rhamnosus or Lactobacillus casei subspecies rhamnosus), Lactobacillus gasseri, Lactobacillus johnsonii, Lactobacillus plantarum and Lactobacillus salivarus. Lactobacillus plantarum 299v strain originates from sour dough. Lactobacillus plantarum itself is of human origin. Other probiotic strains of Lactobacillus are Lactobacillus acidophilus BG2FO4, Lactobacillus acidophilus INT-9, Lactobacillus plantarum ST31, Lactobacillus reuteri, Lactobacillus johnsonii LA1, Lactobacillus acidophilus NCFB 1748, Lactobacillus casei Shirota, Lactobacillus acidophilus NCFM, Lactobacillus acidophilus DDS-1, Lactobacillus delbrueckii subspecies delbrueckii, Lactobacillus delbrueckii subspecies bulgaricus type 2038, Lactobacillus acidophilus SBT-2062, Lactobacillus brevis, Lactobacillus salivarius UCC 118 and Lactobacillus paracasei subsp paracasei F19.

Lactococci that are used or are being developed as probiotics include Lactococcus lactis, Lactococcus lactis subspecies cremoris (Streptococcus cremoris), Lactococcus lactis subspecies lactis NCDO 712, Lactococcus lactis subspecies lactis NIAI 527, Lactococcus lactis subspecies lactis NIAI 1061, Lactococcus lactis subspecies lactis biovar diacetylactis NIAI 8W and Lactococcus lactis subspecies lactis biovar diacetylactis ATCC 13675.

Streptococcus thermophilus is a gram-positive facultative anaerobe. It is a cytochrome-, oxidase- and catalase-negative organism that is nonmotile, non-spore forming and homofermentative. Streptococcus thermophilus is an alpha-hemolytic species of the viridans group. It is also classified as a lactic acid bacteria (LAB). Streptococcus thermophilus is found in milk and milk products. It is a probiotic and used in the production of yoghurt. Streptococcus salivarus subspecies thermophilus type 1131 is a probiotic strain.

Enterococci are gram-positive, facultative anaerobic cocci of the Streptococcaceae family. They are spherical to ovoid and occur in pairs or short chains. Enterococci are catalase-negative, non-spore forming and usually nonmotile. Enterococci are part of the intestinal microflora of humans and animals. Enterococcus faecium SF68 is a probiotic strain that has been used in the management of diarrhoeal illnesses.

The principal probiotic yeast may be Saccharomyces boulardii. Saccharomyces boulardii is also known as Saccharomyces cerevisiae Hansen CBS 5296 and S. boulardii. S. boulardii is normally a non-pathogenic yeast. S. boulardii has been used to treat diarrhoea associated with antibiotic use.

Preferably, the initial cell concentration of the bacteria or fungi in the dried raw material may be in the range of 10⁵ cells to 10¹² cells per gram.

In one embodiment, the biological materials in the composition may be bacterial cells with a cell concentration ranging from 10⁷ to 10¹⁰ cells per gram.

In an alternative embodiment, the biological materials in the composition may be fungal spores with a spore concentration in the range of 10³ cells to 10⁹ cfu/gram.

In a further alternative embodiment, the biological materials in the composition may be fungal mycelia with a mass per volume of 8-33 grams/litre of concentrate.

In one preferred embodiment, the gel mixture produced in step (a) may be allowed to stand at ambient temperature (5° C. to 50° C.) for approximately 5 to 60 minutes, preferably 15-20 minutes before commencing step (b). Further mixing may be completed after standing. The inventors have found that this standing step allows the gel to thicken and increase in viscosity. The standing time also assists in development of the desired thickness or pseudoplastic and even thixotropic properties useful for formation of the first coating.

Preferably, the first coating formed in step (b) may be an approximately uniform thickness of less than 3 mm on at least part of the substrate. In one embodiment, coating may be completed by the step of immersing the substrate into the gel and if required, gently mixing the substrate in the gel to coat the substrate.

In one embodiment, the substrate may be a solid or semi-solid object of an approximately ovoid or spherical shape with a diameter in the range from approximately 0.5 mm to 50 mm. Other shaped substrates may also be used with out departing from the scope of the present invention including discs, chips, flakes or rods.

Preferably, the substrate may be an edible and/or biodegradable solid or semi-solid.

In one embodiment, the substrate materials may be edible materials such as: seeds, prills, pet biscuits, fruits, vegetables, nuts, rice, and dried processed foods such as crackers, cereal grains, pasta, rice and the like.

In further embodiments, the substrate may be clay granules. In one embodiment, the clay granule may be a silicate mineral. Preferably, the clay granule may be an aluminosilicate mineral. One example may be the use of the aluminosilicate mineral zeolite.

In further embodiments, the substrate may be biopolymer beads. Examples of biopolymer beads include polyhydroxyalkanoate beads and agarose beads.

Mixtures of the above may also be used with out departing from the scope of the invention.

Preferably, the desiccation agent or agents may be used to reduce the gel water activity. This not only helps to stabilise the biological material but also helps to make the eventual product easier to handle by reducing the coated substrate ‘stickiness’. It is understood that the desiccation agent or agents absorb aqueous solution from the gel coating in order to reduce the water activity. The agent or agents owing to their desiccation properties remain dry to touch even after coating and absorption.

In preferred embodiments, the desiccation agent or agents may be selected from the group including: celite, talc, bentonite, zeolite, rice powder, potato starch, corn starch, lactose, sucrose, glucose, mannitol, sorbitol, calcium carbonate, silicone dioxide, calcium phosphate, celluloses, polyethylene glycol, and combinations thereof. It should be appreciated by those skilled in the art that the above list is provided by way of example and that desiccation agents of the art in general may be added depending on the end application e.g. food applications require food safe agents.

Preferably, the desiccation agent may be a fine powder with a particle size less than 1 mm, more preferably less than 100 μm.

Preferably, the amount of desiccation agent or agents used may range from 1 part biopolymer to between 1 and 5 parts desiccation agent or agents. In one preferred embodiment the ratio is approximately 1 part biopolymer to 2 parts desiccation agent.

In one embodiment, the desiccation agent or agents may be pre-dried before use in the above process to reduce the initial water activity of the desiccation agent or agents. Preferably, the pre-dried desiccant water activity may be approximately 0.1.

The inventors have found that once desiccation agent has been added, the resulting double coating on the substrate has a low water activity. In practice this water activity may be less than at least 0.7 and more preferably, is less than approximately 0.4. It should be appreciated that this may be a very low water activity and the method therefore provides a highly stable environment for the microbial material without the need to perform a separate drying step.

In one embodiment, the stabilised biological material and substrate may also include at least one oil. Preferably, where used, the oil may be added during step (a) of the method and before addition of biological material. Oil has been found by the inventors to assist with homogeneity of the mixture and prevents clumping, localised non-mixing and improves dispersion.

In one embodiment, the oil may be edible oil.

Preferred oils may be vegetable based oils. Alternatively, oils may be marine based such as fish or seaweed based oils. Combinations of oils may also be used.

In a preferred embodiment the oil used may have high levels of antioxidants such as but not limited to, cold pressed virgin oils.

More specifically, the oil may be selected from: olive oil, canola oil, sunflower seed oil, hydrolyzed oils, and combinations thereof.

In one preferred embodiment, the oil may be olive oil although it should be appreciated that other oils may be used with similar chemical and physical characteristics without departing from the scope of the invention.

Preferably, the ratio of biopolymer to oil mixed in step (a) may be in the range 1:10 to 10:1 by weight. In a more preferred embodiment, the ratio of dried biological material to oil may be from 1:1 to 1:4. In a yet more preferred embodiment the ratio may be approximately 1:1.

In one embodiment, the stabilised biological material and substrate may also include at least one antioxidant substance. Preferably, where used, the antioxidant may be added during step (a) of the method. Preferred antioxidant substances include: tocopherol, ascorbic acid, and combinations thereof.

In one embodiment, the stabilised biological material and substrate may also include at least one surfactant compound. In one embodiment the surfactant may be mixed with the biological material to form the raw aqueous biological concentrate. In one embodiment the surfactant may have a hydrophilic moiety. In one preferred embodiment, the surfactant may be Triton X-100™. Note that this surfactant may be used with or without oil being present in the composition.

Preferably, the term ‘ambient’ refers to normal room temperatures, humidity's and atmospheric pressure. More specifically, this term refers to a temperature ranging from approximately 10° C. to 50° C., more preferably 15 to 25° C., and a relative humidity ranging from 0% to 70%, more preferably 40-80% and standard atmospheric pressure.

Preferably, the composition produced may be stored in a sealed environment. By way of example, the composition may be stored in bags or sealed polystyrene containers. This is to help protect the composition from attack by humidity or oxidative degradation.

An advantage found by the inventors is that the composition does not need to be vacuum sealed. Unlike prior art methods, removal of oxygen from a container prior to sealing is not essential and has a negligible effect on viability.

Preferably, the above method may be completed under ambient conditions. As may be appreciated, this is a key advantage as the process does not need to be completed under special temperature, humidity or inert atmospheres unlike prior art methods. By way of example the inventors have found good process efficiencies where the efficiency is a percentage measure between levels of viable cells before and after processing.

For use, the coated substrate may simply placed into the environment. For example, in an agricultural application, a coated seed is drilled into the soil and the aqueous environment surrounding the seed breaks down the coating layer releasing the biological agent such as an antifungal agent into the surrounding environment. In an alternative example, the substrate may be a cereal grain such as a bran flake which is coated with probiotic microbes. On ingestion, the aqueous environment within the gut causes the coating to breakdown releasing the probiotic agent into the gut.

It is the inventor's experience that the above method and product lends itself well to large scale processing as it avoids the need to use slow and energy intensive physical drying methods such as air, spray or freeze drying. By contrast, the method and product of the present invention uses a ‘chemical’ drying step by addition of desiccation agent or agents.

In addition, the product is ideally suited for mass distribution as it is in a form ready for delivery including the substrate and does not need any special treatment prior to application such as re-hydration and/or mixing. Prior art methods tend to require a re-hydration step before application which is undesirable especially when delivery is on a large scale, due to the extra labour and handling required, as well as the danger of losing viable biological material.

It should be appreciated from the above description that there is provided a method and coated substrate product that offers considerable advantages over the prior art including:
The ability to stabilise, store and then utilise the biological material at a later date;
The ability to deliver both the biological material and substrate in one product;
Removal of the need to complete any extra handling steps prior to application of the biological material and substrate such as re-hydration;
Removal of the need to physically dry the biological material;
An extremely low residual water activity and hence high stability environment;
A more practical method for producing and marketing of large quantities of stabilised microbial material.

### BRIEF DESCRIPTION OF THE DRAWINGS

Further aspects of the present invention will become apparent from the following description, which is given by way of example only and with reference to the accompanying drawings in which:

FIG. 1 shows a flow diagram of the process;

FIG. 2 shows a graph illustrating the viability of formulations 1-3 over time at 25° C.;

FIG. 3 shows a graph illustrating the viability of formulations 4-6 over time at 25° C.;

FIG. 4 shows a graph illustrating the viability of formulations 7-8 over time at 25° C.;

FIG. 5 shows a graph illustrating the viability of formulations 4-6 over time at 20° C.;

FIG. 6 shows a graph illustrating the viability of formulation 7 over time at 20° C.;

FIG. 7 shows a graph illustrating the viability of formulation 9 over time at 20° C.;

FIG. 8 shows a graph illustrating the viability of formulation 10 over time at 20° C.;

FIG. 9 shows a graph illustrating the viability of formulation 13 over time at 25° C.;

FIG. 10 shows a graph illustrating the viability of formulation 14 over time at 30° C.;

FIG. 11 shows a graph illustrating the viability of formulation 15 over time at 30° C.;

FIG. 12 shows a graph illustrating the viability of formulation 16 over time at 30° C.; and,

FIG. 13 shows a graph illustrating the viability of a Lactobacillus formulation at 30° C. where different ratios of desiccant to biopolymer are tested. Formulations are labelled as follows; ‘6<#1><#2>4’ where #1=‘n’ or ‘y’ referring to whether or not the formulation was air dried or not and #2=2% or 4% biopolymer concentration. Each column per formulation represents a one month time interval.

### BEST MODES FOR CARRYING OUT THE INVENTION

Preferred best methods for producing the product of the present invention and uses of these products are now described.

### Example 1

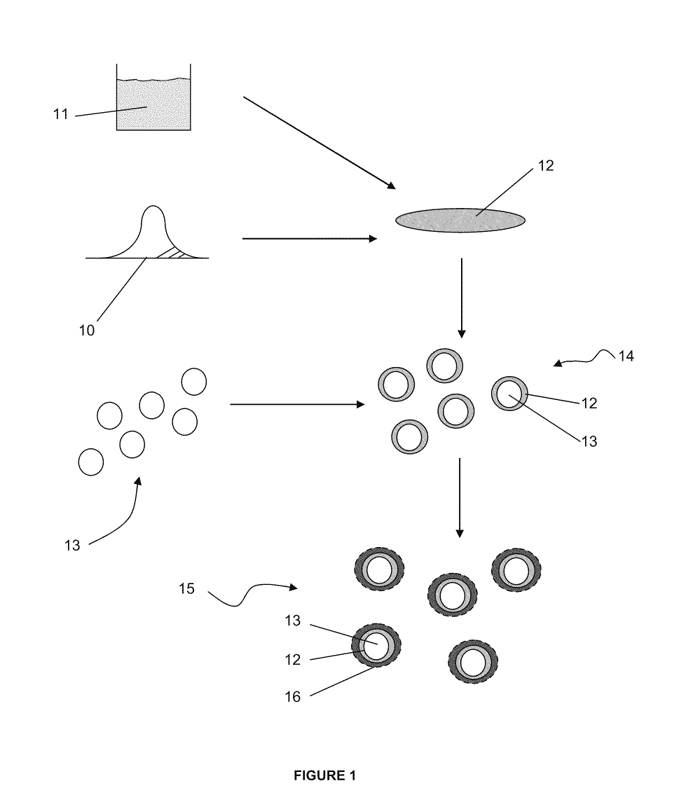

A general method of manufacturing the product of the present invention is described with reference to FIG. 1.

Initially mix dry and powdered gum (biopolymer) with oil at room temperature so that the mixture forms a coarse granular mixture 10.

Prepare an aqueous based concentrate of biological material 11 and mix this with the gum and oil mixture 10 to form a gel 12.

Optionally, allow the gel 12 to stand for 5-60 minutes.

Form a first coating on the substrate 13 with the gel 12 in one option by dipping the substrate 13 into the gel 12 to form a first coated substrate 14.

Subsequently add a second coat 16 to the first coated substrate 14 to form a double coated substrate 15.

As should be appreciated no oils or other agents are added in the above general method. The inventors have found that this basic process may be sufficient to stabilise the biological material. Oils and other substances may optionally be added and these are discussed further below.

### Example 2

Various formulations are now described in Table 1 below being various combinations for producing the composition of the invention:

TABLE 1
Composition Combinations

| Formulation | | Biological | | | 2^{nd} Coating |
|---|---|---|---|---|---|
| Number | Substrate | Material | Gum | Oil | Desiccant(s) |
| 1 | Clover seed, | Rhizobium | Xanthan, | Salad and | Talc |
| | Var. Huia | leguminosarum | Locust | cooking oil | |
| | | | Bean | | |
| 2 | Clover seed, | Rhizobium | Xanthan, | Salad and | Talc |
| | Var. Huia | leguminosarum | Guar, | cooking oil | |
| | | | Locust | | |
| | | | Bean | | |
| 3 | Clover seed, | Rhizobium | Xanthan | Salad and | Talc |
| | Var. Huia | leguminosarum | | cooking oil | |
| 4 | Bran | Lactobacillus | Xanthan, | Olive oil | Rice powder |
| | | acidophilus | Guar, | | |
| | | | Locust | | |
| | | | Bean | | |
| 5 | Bran | Lactobacillus | Locust | Olive oil | Rice powder |
| | | acidophilus | Bean, | | |
| | | | Guar | | |
| 6 | Bran | Lactobacillus | Xanthan | Canola oil | Rice powder |
| | | acidophilus | | | |
| 7 | Bran | Bifidobacterium | Xanthan, | Olive oil | Rice powder, |
| | | lactis | Guar, | | and potato |
| | | | Locust | | starch |
| | | | Bean | | |
| 8 | Bran | Bifidobacterium | Xanthan, | Olive oil | Rice powder, |
| | | lactis | Guar, | | potato starch |
| | | | Locust | | |
| | | | Bean | | |
| 9 | Zeolite | Serratia | Xanthan | Salad and | Bentonite and |
| | | entomophila | | cooking oil | talc |
| 10 | Zeolite | Beauveria | Xanthan | No oil | Bentonite and |
| | | bassiania | | | talc |
| 11 | Carrot seed | Serratia | Xanthan | Salad and | Bentonite and |
| | | entomophila | | cooking oil | Talc |
| 12 | Onion seed | Pseudomonas | Xanthan, | Salad and | Bentonite and |
| | | | Guar, | cooking oil | Talc |
| | | | Locust | | |
| | | | Bean | | |
| 13 | Bran | Lactobacillus | Xanthan | Olive oil | Rice powder |
| | | rhamnosus | | | |
| 14 | Bran | Lactobacillus | Xanthan | Olive oil | Rice powder |
| | | casei | | | |
| 15 | Bran | Lactobacillus | Locust | Olive oil | Rice powder |
| | | rhamnosus | bean, guar | | |
| 16 | Bran | Lactobacillus | Locust | Olive oil | Rice powder |
| | | casei | bean, guar | | |

### Example 3

A detailed methodology is now described to produce Formulation 2 (and variations used to make Formulations 1 and 3):
(a) Rhizobium leguminosarum biovar trifolii (CC275e) was produced using a modified yeast malt extract broth and further processed to form a concentrate.
(b) 3 grams xanthan gum, 1 gram guar gum and 1 gram locust bean gum were mixed together.
(c) 0.5 grams of salad and cooking oil was then added to the gum mixture
(d) The mixture from step (c) was then combined with the concentrate of step (a) to form a gel.
(e) 2 grams of gel was coated onto 44 grams of white clover seed (variety Huia) and the gel and clover seed mixed to obtain a uniform coating on seed surface (a ‘first coating’).
(f) A second coating of 4 grams of talc (desiccant) was added to the first coated seed resulting in a single double coated flowable seed.
(g) Coated seeds were bagged in thick gas transferable bag (120 μm thickness) and stored.

The same methodology was used for Formulations 1 and 3 except the gum was varied in each case with Formulation 1 using xanthan and locust bean gum only and Formulation 3 using only xanthan gum.

### Example 4

A detailed methodology is now described to produce Formulation 5 (and variations used to make Formulation 4):
(a) Frozen cells of Lactobacillus acidophilus were obtained from a commercial source and held in sealed containers at −80° C.
(b) 0.25 grams of locust bean gum and 0.25 grams of guar gum were mixed with 0.5 grams of extra virgin olive oil.
(c) 11.5 ml of L. acidophilus concentrate was added to the mixture of step (b) to form a gel.
(d) 15 μl of antioxidant (mixed tocopherol) was added to the gel of step (c).
(e) 1.6 grams of gel was coated (first coating) onto pre-dried bran (substrate dried at 80° C. for 24 hrs) and the gel and bran mixed to achieve a uniform coating on the bran.
(f) 1.6 grams of pre-dried rice powder (dried at 80° C. for 24 hrs) was then added and mixed onto the first coating (being the second coating).
(g) 5 gram samples were placed in foil sachets and stored.

The measured water activity after formulation was a_{w} 0.479.

Formulation 4 was made using the same method as for Formulation 5 except that xanthan gum was also used in addition to locust bean and guar gum.

### Example 5

A detailed methodology is now described to produce Formulation 6:
(a) Frozen cells of Lactobacillus acidophilus were obtained from a commercial source and held in sealed containers at −80° C.
(b) 0.5 grams of xanthan gum was mixed with 0.5 grams of canola oil.
(c) 11.5 ml of thawed L. acidophilus concentrate was added to the mixture of step (b) which on mixing formed a gel.
(d) 15 μl of antioxidant (mixed tocopherol) was then added to the gel.
(e) 1.6 grams of the gel was then coated (first coating) onto pre-dried bran (substrate dried at 80° C. for 24 hrs) and the gel and bran mixed to achieve a uniform coating on the bran.
(f) 1.6 grams of pre-dried rice powder (dried at 80° C. for 24 hrs) was then added and mixed onto the first coating (being the second coating).
(g) 5 gram samples were placed in foil sachets and stored.

The measured water activity after formulation was a_{w} 0.525.

### Example 6

A detailed methodology is now described to produce Formulation 7:
(a) Frozen cells of Bifidobacterium lactis were obtained from a commercial source and held in sealed containers at −80° C.
(b) 0.208 grams of xanthan gum, 0.208 grams of locust bean gum and 0.208 grams of guar gum were mixed with 0.625 grams of extra virgin olive oil.
(c) 0.0125 grams of ascorbic acid (acting as an antioxidant) was added to the mixture of step (b).
(d) 11.25 ml of B. lactis diluted concentrate (diluted in 0.15% Bactopeptone) was then added to the mixture of step (c) to form a gel.
(e) 1.6 grams of the gel was coated (first coating) onto pre-dried bran (substrate dried at 80° C. for 24 hrs) and the gel and bran mixed to achieve a uniform coating on the bran.
(f) 1.6 grams of pre-dried rice powder and potato starch (Pacelli BC) were then mixed together at a 1:1 ratio where the powder and starch were dried at 80° C. for 24 hrs prior to mixing. The powder and starch mixture was then coated onto the first coating (being the second coating).
(g) 5 gram samples were placed in foil sachets and stored

The measured water activity after formulation was a_{w} 0.411.

### Example 7

A detailed methodology is now described to produce Formulation 8:
(a) Frozen cells of Bifidobacterium lactis were obtained from a commercial source and held in sealed containers at −80° C.
(b) 0.208 grams of xanthan gum, 0.208 grams of locust bean gum and 0.208 grams of guar gum were mixed with 0.625 grams of extra virgin olive oil.
(c) 0.0125 grams of ascorbic acid (acting as an antioxidant) was added to the mixture of step (b).
(d) 11.25 ml of B. lactis diluted concentrate (diluted in 0.15% Bactopeptone) was added to the mixture of step (c) and a gel formed.
(e) 1.6 grams of the gel was then coated (first coating) onto pre-dried bran (dried at 80° C. for 24 hrs) and the gel and bran mixed to achieve a uniform coating on the bran.
(f) 1.6 grams of pre-dried rice powder and potato starch (Pasellii BC) were then mixed together at a 1:1 ratio where the powder and starch were dried at 80° C. for 24 hrs prior to mixing. The powder and starch mixture was then coated onto the first coating (being the second coating).
(g) 5 gram samples were placed in foil sachets and stored.

The measured water activity after formulation was a_{w} 0.411.

### Example 8

A detailed methodology is now described to produce Formulation 9:
(a) Serratia entomophila bacteria was obtained from a commercial source and formed into a broth.
(b) 15 grams of xanthan gum was mixed with 15 grams of salad and cooking oil.
(c) 230 ml of broth from step (a) was added to the mixture of step (b) and mixed thoroughly to form a gel.
(d) The gel was coated on 650 grams of zeolite granules (2-4 μm size) and mixed to form a uniform coating on the zeolite (first coating).
(e) 50 grams of bentonite and talc mixed at a 1:1 ratio was then coated onto the first coating (being a second coating).
(f) A further 50 grams of talc was then added to achieve a single flowable particle of zeolite.

The measured water activity after formulation was a_{w} 0.989.

Formulations 11 and 12 were made using the same method as for Formulation 9 except that the substrate was changed to carrot seed in Formulation 11 and onion seed in Formulation 12.

### Example 9

A detailed methodology is now described to produce Formulation 10:
(a) B. bassiania spores were obtained from a commercial source and stored at 4° C. until use.
(b) 4 grams of xanthan gum was mixed with 158 grams of distilled water.
(c) 28 grams of spores were then mixed with 163 grams of 0.05% Triton X-100 dispersing agent and homogenised using polytron.
(d) The xanthan suspension of step (b) was then mixed with the spore suspension of step (c) to form a homogeneous gel.
(e) The gel was then coated (first coating) onto 845 grams of zeolite granules (2-4 μm) and the gel and zeolite mixed to form a uniform coating.
(f) 65 grams of bentonite and talc mixed at a 1:1 ratio was then added coated onto the first coating (being a second coating).
(g) Two additional coatings using talc alone were then completed.
(h) Samples were then packed in gas transferable bag (80 μm thick) and stored.

In a further embodiment, oil may also be added in step (b) although this is not essential.

### Example 10

A detailed methodology is now described to produce Formulations 13 and 14:
a) Weigh 0.2 grams of locust bean gum and 0.2 grams of guar gum and mix to form a gum mixture.
b) To the gum mixture add 0.4 grams olive oil and mix.
c) Add to the gum mixture and olive oil mixture a prepared cell concentrate to a final volume of 10 ml.
d) Next add 12 μL of vitamin E at which point a gel is produced
e) To 70 grams of bran dried at 80° C. for 24 hours coat 2.8 grams of gel onto the bran with the aid of gentle mixing.
f) To the coated bran coat 2.8 grams of rice powder dried at 80° C. for 24 hours and disperse with the aid of gentle mixing.

### Example 11

A detailed methodology is now described to produce Formulations 15 and 16:
a) Weigh 0.4 grams of xanthan gum.
b) To the gum add 0.4 grams olive oil and mix.
c) Add to the gum and olive oil mixture a prepared cell concentrate to a final volume of 10 ml.
d) Next add 12 μL of vitamin E at which point a gel is formed.
e) To 70 grams of bran dried at 80° C. for 24 hours coat 2.8 grams of gel onto the bran with the aid of gentle mixing.
f) To the coated bran coat 2.8 grams of rice powder dried at 80° C. for 24 hours and disperse with the aid of gentle mixing.

### Example 12

The stability of the double coated substrate is now shown over the short term at a slightly higher temperature of 25° C.

In each stability test, the shelf life was monitored at monthly intervals and tested via standard protocols to measure viability.

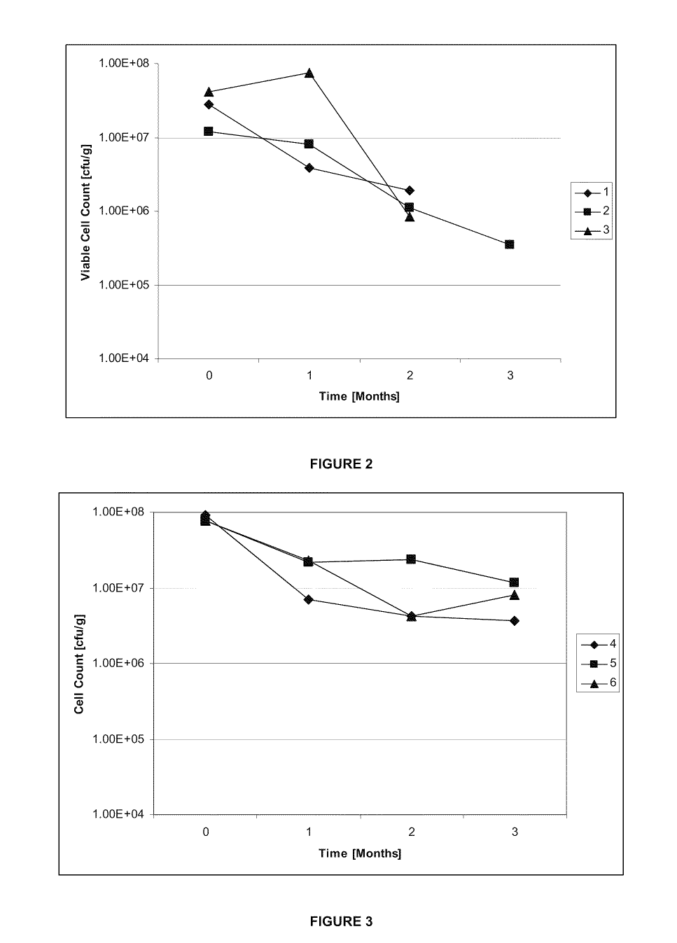

FIG. 2 shows the viability of Formulations 1, 2, and 3 when stored over time at 25° C. As can be seen, the reduction in viability is less then 2 log losses over 3 months of storage.

FIG. 3 shows the viability of Formulations 4, 5, and 6 when stored over time at 25° C. As can be seen, the reduction in viability is also less then 2 log losses over 3 months of storage.

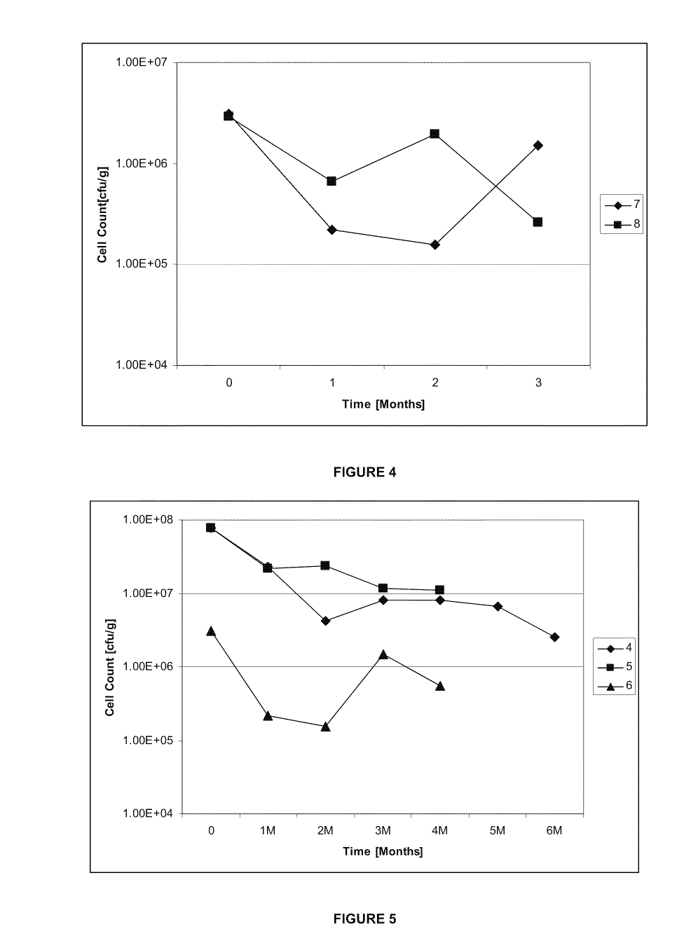

FIG. 4 shows the viability of Formulations 7 and 8 when stored over time at 25° C. As can be seen, the reduction in viability is also less then 2 log losses over 3 months of storage.

### Example 13

Long term survival is now shown based on further trials completed by the inventors using a storage temperature of 20° C. The samples collected were tested using similar standard protocols as for Example 12.

FIG. 5 shows the viability of Formulations 4, 5, and 6 when stored at 20° C. for up to 6 months. Typically the loss in viability is less than 1 log loss and never greater than 2 logs.

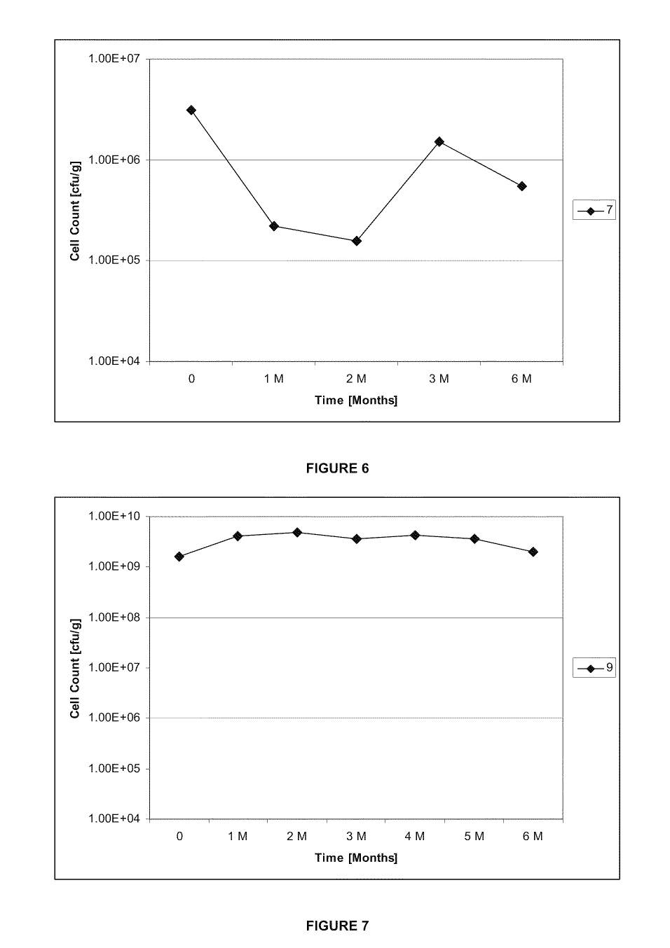

FIG. 6 shows the viability of Formulation 7 when stored at 20° C. for 6 months. As above, the loss in viability is less than 1 log loss and never greater than 2 logs.

FIG. 7 shows the viability for Formulation 9 when stored at 20° C. for 6 months. In this example, the viability remains well within 1 log loss.

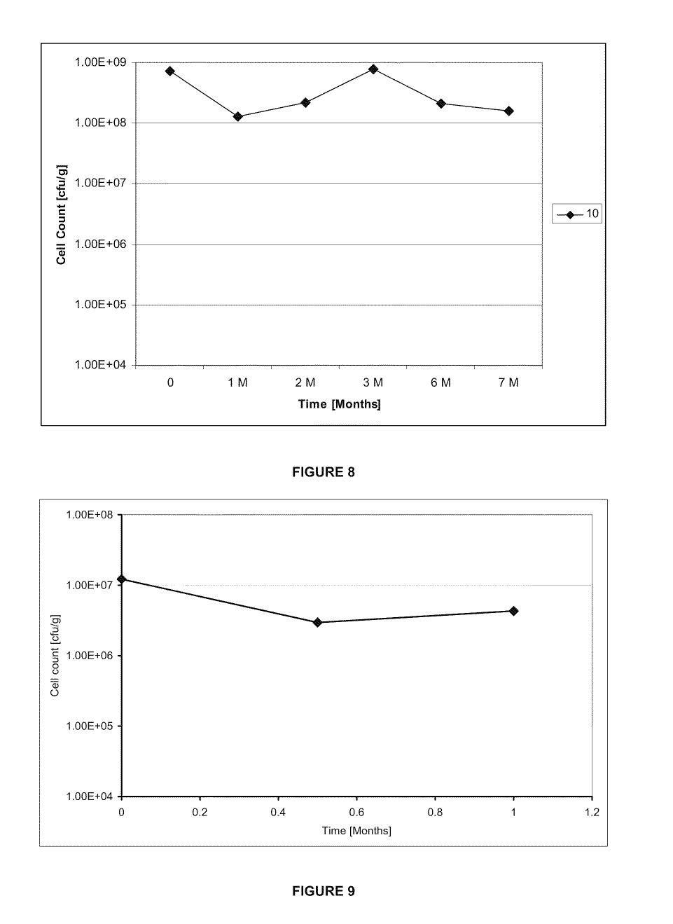

FIG. 8 shows the viability of Formulation 10 when stored at 20° C. for 7 months. In this case, the viability also did not decrease more than 1 log loss.

### Example 14

Long term survival is now shown based on further trials completed by the inventors using a storage temperature of 25° C. The samples collected were tested using similar standard protocols as for Example 12.

FIG. 9 shows the viability of Formulation 13 when stored at 25° C. for up to 1 month. Typically the loss in viability is less than 1 log loss.

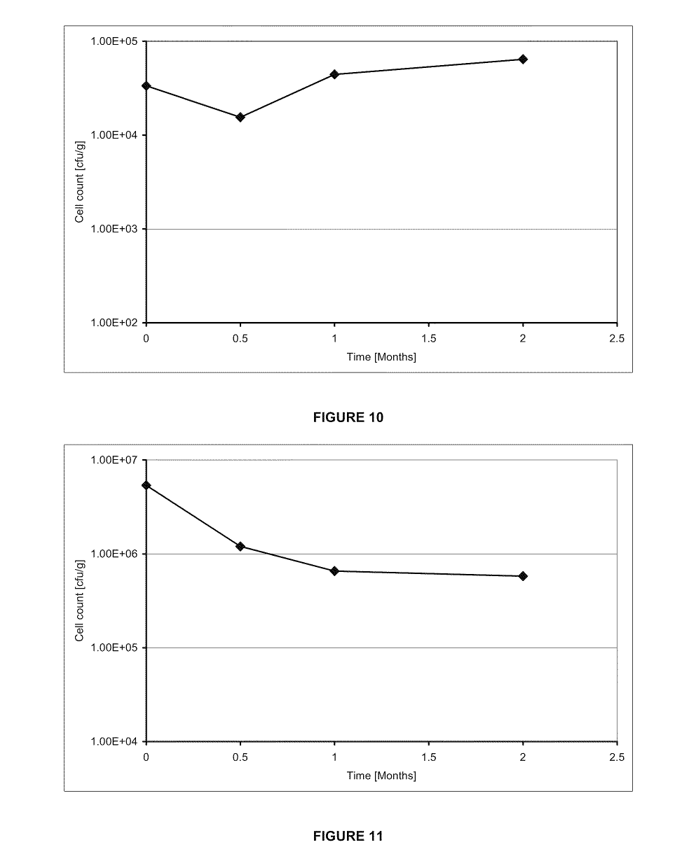

FIG. 10 shows the viability of Formulation 14 when stored at 30° C. for up to 2 months. Typically the loss in viability is less than 1 log loss.

FIG. 11 shows the viability of Formulation 15 when stored at 30° C. for up to 2 months. Typically the loss in viability is less than 1 log loss.

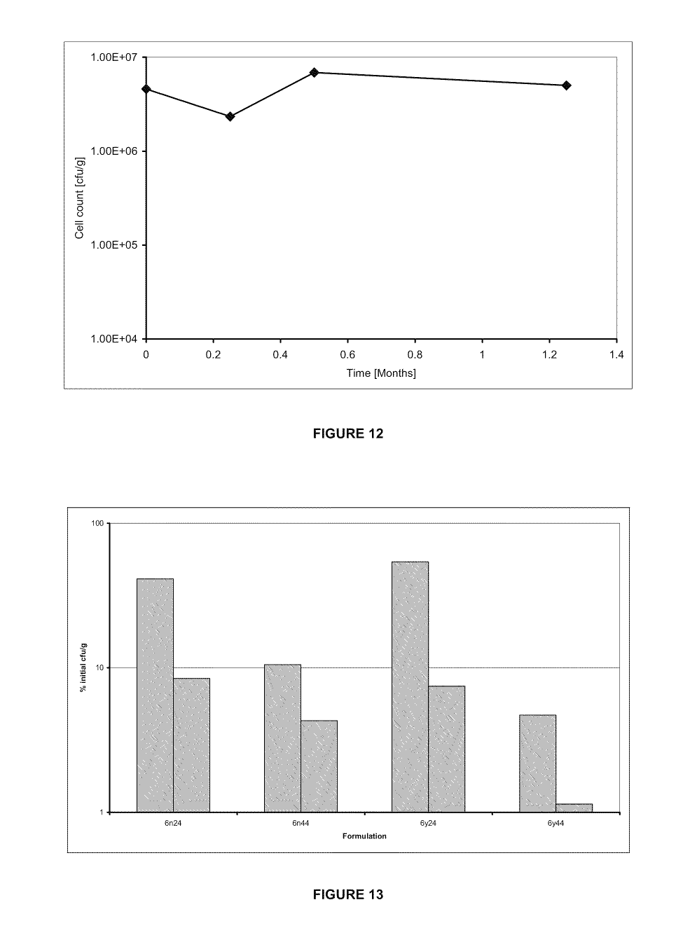

FIG. 12 shows the viability of Formulation 16 when stored at 30° C. for up to 2 months. Typically the loss in viability is less than 1 log loss.

### Example 15

In this example, an experiment is described to show the impact that the ratio of desiccant to biopolymer has on stability.

The experiment was completed by preparing various formulations including different desiccant to biopolymer mixtures by the steps of:
(a) Mixing together freshly collected Lactobacillus acidophilus cells diluted at a 1:1 ratio with 0.15% bactopeptone (pH 7.2);
(b) separately weighing out 0.2 g of locust bean gum and 0.2 g of guar gum or 0.4 g of locust bean gum or 0.4 g of guar gum.
(c) Adding either 0.4 g or 0.8 g of olive oil to the biopolymer mixture of step (b) and mixing.
(d) Adding the biopolymer and olive oil mixture of step (c) to the prepared cell concentrate of step (a) to a final volume of 10 mL.
(e) Adding 12 μL of vitamin E to the mixture of step (d).
(f) Coating 70 grams of bran flakes with sufficient mixture of step (e) with the aid of gentle mixing.
(g) Adding 2.8 grams of rice powder and dispersing this with the aid of gentle mixing.
(h) Packaging the resulting product of step (g) in vacuum sealed foil after storage at 30° C. over a saturated MgCl₂ solution for 6 days.

Samples were subsequently stored at 30° C. and monitored for cfu/g and water activity at t=0 and after one month.

As shown in FIG. 13, the stability of the resulting formulation decreases as the amount of biopolymer increases (and the amount of desiccant decreases in proportion).

### Example 16

This example describes a method of producing a stabilised probiotic culture coated onto bran flakes.

The method involves the steps of:
(a) Mixing together freshly collected cells diluted at a 1:1 ratio with 0.15% bactopeptone (pH 7.2)
(b) Separately weighing 0.2 grams of locust bean gum and 0.2 grams of guar gum and combining both gums.
(c) Adding 0.4 grams of olive oil to the gum mixture of step (b).
(d) Adding the gum and olive oil mixture of step (c) to the prepared cell concentrate of step (a) to a final volume of 10 mL.
(e) Adding 12 μL of vitamin E.
(f) To 70 grams of bran add sufficient mixture of step (e) to coat the bran evenly with.
(g) Mix the coated bran produced from step (f) with 2.8 grams of rice powder.

### Example 17

This example describes two further product mixtures using the stabilised probiotic composition of the present invention.

Fruit Bar

| | |
|---|---|
| Dates | 40 grams |
| Raisins | 40 grams |
| Figs | 40 grams |
| Oats | 20 grams |
| Stabilized probiotic culture on bran from Example 16 | 20 grams |

Breakfast cereal

| | |
|---|---|
| Whole wheat flour | 10 grams |
| Brown Sugar | 10 grams |
| Coconut | 10 grams |
| Pecans | 7.5 grams |
| Wheat germ | 10 grams |
| Oil | 7.5 grams |
| Raisins | 10 grams |
| Stabilized probiotic culture on bran from Example 16 | 35 grams |

It should be appreciated from the above examples that there is provided a method and products that stabilise biological materials so that they may be stored for significant periods of time (up to 7 months or more). Because the biological material is incorporated with a substrate, the product resulting is ready for use in various applications such as in foods and agriculture, removing the need for extra handling steps.

Aspects of the present invention have been described by way of example only and it should be appreciated that modifications and additions may be made thereto without departing from the scope thereof as defined in the appended claims.

1-55. (canceled)

56. A composition including:
(a) a substrate;
(b) a first coating that at least partially coats the surface of the substrate including: at least one gum based biopolymer; and an aqueous concentrate of biological material comprising a microorganism; and,
(c) a second coating that at least partially coats the surface of the first coating including at least one desiccation agent.

57. The composition as claimed in claim 56 wherein the composition includes at least one further coating that at least partially coats the surface of the second or later coating wherein the further coating(s) include at least one desiccation agent.

58. The composition as claimed in claim 56 wherein the composition is sufficiently stable such that no more than a 2 log loss in biological viability occurs when the composition is stored for at least 1 month at 20° C. to 30° C.

59. The composition as claimed in claim 56 wherein the composition has a water activity of less than 0.5 or the composition is dry to touch.

60. The composition as claimed in claim 56 wherein the biopolymer gum has a molecular weight of between 5000 and 50 million or the biopolymer gum is selected from agar, alginate, cassia, dammar, pectin, beta-glucan, glucomannan, mastic, chicle, psyllium, spruce gum, xanthan gum, gellan gum, acacia gum, guar gum, locust bean gum, carrageenans, gum arabic, karaya gum, ghatti gum, tragacanth gum, konjac gum, tara gum, and combinations thereof.

61. The composition as claimed in claim 56 wherein the concentration of biopolymer or biopolymers in the composition is approximately 1% to 10% by weight.

62. The composition as claimed in claim 56 wherein the biological material comprises probiotic bacteria or the biological material is selected from the genera: Serratia, Xanthamonas, Pseudomonas, Rhizobium, Beauveria, Metarhizium, Bifidobacterium, Lactobacillus, Streptococcus (Enterococcus), Yersinia, Trichoderma, and combinations thereof.

63. The composition as claimed in claim 56 wherein the substrate is selected from edible materials, clays, biopolymer beads, and combinations thereof.

64. The composition as claimed in claim 56 wherein the desiccation agent or agents are selected from powdered clays or powdered carbohydrate materials, or are selected from celite, talc, bentonite, zeolite, rice powder, potato starch, corn starch, lactose, sucrose, glucose, mannitol, sorbitol, calcium carbonate, silicone dioxide, calcium phosphate, celluloses, polyethylene glycol, and combinations thereof.

65. The composition as claimed in claim 56 wherein the composition also includes at least one oil, at least one edible oil, at least one vegetable oil, or an oil selected from olive oil, canola oil, sunflower seed oil, hydrolyzed oils, and combinations thereof.

66. A food, nutraceutical product, or food ingredient including a composition as claimed in claim 56.

67. A method of producing a composition including stabilised biological material and a substrate, the method comprising:
(a) mixing at least one substantially dry and powdered biopolymer with an aqueous concentrate of biological material comprising a microorganism to form a gel;
(b) coating the gel formed in step (a) as a first coating onto at least part of the surface of a substrate material to form a gel coated substrate; and,
(c) at least partially coating the first coating with at least one desiccation agent to form a second coating.

68. The method as claimed in claim 67 wherein the second coating is coated with at least one further coating wherein the further coating(s) include at least one desiccation agent.

69. The method as claimed in claim 67 wherein the composition after step (c) is sufficiently stable such that no more than a 2 log loss in biological viability occurs when the composition is stored for at least 1 month at 20° C. to 30° C.

70. The method as claimed in claim 67 wherein the composition after step (c) has a water activity of less than 0.5 or is dry to touch.

71. The method as claimed in claim 67 wherein the biopolymer gum has a molecular weight of between 5000 and 50 million or is selected from agar, alginate, cassia, dammar, pectin, beta-glucan, glucomannan, mastic, chicle, psyllium, spruce gum, xanthan gum, gellan gum, acacia gum, guar gum, locust bean gum, carrageenans, gum arabic, karaya gum, ghatti gum, tragacanth gum, konjac gum, tara gum, and combinations thereof.

72. The method as claimed in claim 67 wherein the concentration of biopolymer or biopolymers in the composition after step (c) is approximately 1-10% by weight.

73. The method as claimed in claim 67 wherein the biological material comprises probiotic bacteria or the biological material is selected from the genera: Serratia, Xanthamonas, Pseudomonas, Rhizobium, Beauveria, Metarhizium, Bifidobacterium, Lactobacillus, Streptococcus (Enterococcus), Yersinia, Trichoderma, and combinations thereof.

74. The method as claimed in claim 67 wherein the gel mixture produced in step (a) is allowed to stand at ambient temperature for approximately 5 to 60 minutes before commencing step (b).

75. The method as claimed in claim 67 wherein the first coating formed in step (b) is an approximately uniform thickness of less than 3 mm on the substrate.

76. The method as claimed in claim 67 wherein the substrate material used in step (c) is selected from seeds, clay granules, prills, pet biscuits, foods such as fruits, vegetables, nuts, rice and dried processed foods such as crackers, cereal grains, and combinations thereof.

77. The method as claimed in claim 67 wherein the desiccation agent or agents used in step (c) are a fine dry powder with a particle size less than 1 mm or wherein the desiccation agent or agents are dry powdered materials selected from celite, talc, bentonite, zeolite, rice powder, potato starch, corn starch, lactose, sucrose, glucose, mannitol, sorbitol, calcium carbonate, silicone dioxide, calcium phosphate, celluloses, polyethylene glycol, and combinations thereof.

78. The method as claimed in claim 67 wherein the amount of desiccation agent or agents used ranges from 1 part biopolymer to between 1 and 5 parts desiccation agent or agents.

79. The method as claimed in claim 67 wherein the composition also includes at least one oil, at least one edible oil, at least one vegetable oil, or an oil selected from olive oil, canola oil, sunflower seed oil, hydrolyzed oils, and combinations thereof.

80. The method as claimed in claim 67 wherein the ratio of biopolymer to oil mixed in step (a) is in the range 1:10 to 10:1 by weight.

81. A product, food, nutraceutical product, or food ingredient including a composition produced by the method as claimed in claim 67.
